(19) United States
(12) Plant Patent Application Publication
(10) Pub. No.: US 2023/0132405 P1
(43) Pub. Date: Apr. 27, 2023

(54) Cannabis plant named 'OT-1'

(50) ;

(71) Applicant: Matthew Gordon, Oak Harbor, WA (US)

(72) Inventor: Matthew Gordon, Oak Harbor, WA (US)

(21) Appl. No.: 17/803,798

(22) Filed: Nov. 29, 2022

Related U.S. Application Data
(63) Continuation-in-part of application No. 17/119,215, filed on Dec. 11, 2020, now abandoned.
(60) Provisional application No. 62/947,516, filed on Dec. 12, 2019.

(51) Int. Cl. A01H 6/28

(52) U.S. Cl. PLT/258

(57) ABSTRACT

The unique annual herbaceous Cannabis plant variety ‘OT-1’ is provided. The variety is a selection intended for use as medical oil, and medicinal herb for sale in Cannabis dispensaries and as a home garden plant.

### CROSS REFERENCE TO RELATED APPLICATIONS

The present Application for Patent is a continuation-in-part of U.S. Application No. 17/119,215 filed on Dec. 11, 2020, which in turn claims the benefit of provisional patent Application No. 62/947,516 filed on Dec. 12, 2019, each of which is hereby, expressly incorporated by reference herein.

Latin name of the genus and species:

Genus—Cannabis.

Species—sativa.

Variety denomination:

The new Cannabis plant claimed is of the variety denominated ‘OT-1’.

### BACKGROUND OF THE INVENTION

### Field of the Invention

The present invention relates to a new and distinct annual variety of C. sativa hybrid, which has been given the variety denomination of ‘OT-1’. ‘OT-1’ is intended for use as medical oil, and medicinal herb for sale in Cannabis dispensaries and as a home garden plant.

### Background of the Related Art

The genus Cannabis has been in use by humans for millennia, due to the multiplicity of its benefits to humans, including the considerable value and utility of its fiber, the nutritional value of its seeds, and the medicinal value of its floral parts and products made from them. Currently the genus is under intense legal commercialization in the United States as industrial hemp for a variety of purposes including biodegradable plastics and building materials, clothing, paper, food, fuel, and medicines.

Cannabidiol (CBD) extracted from Cannabis is widely used in over-the-counter medicines and topical treatments and is also the active ingredient in the FDA-approved drug Epidiolex®. CBD is just one of at least dozens—perhaps hundreds—of cannabinoids endogenous to Cannabis, tetrahydrocannabinol (THC) being the other cannabinoid that is most well-known. The cannabinoids as a group interact with the human endocannabinoid receptors, which are distributed in the brain and throughout the body. The study of the endocannabinoid system (ECS) in humans and other mammals is an area of increasing interest and holds tremendous promise for the future of medicine. See, e.g., Russo (2019). Cannabis and Pain, Pain Medicine, 20(10): 1;20(11):2083-2085; and Russo (2016). Clinical Endocannabinoid Deficiency Reconsidered: Current Research Supports the Theory in Migraine, Fibromyalgia, Irritable Bowel, and Other Treatment-Resistant Syndromes, Cannabis Cannabinoid Res. 1(1): 154-165.

Non-hemp forms of Cannabis, frequently referred to as marijuana, have been legalized for medicinal use in many states and for recreational use (sometimes called “adult use”) in a growing number of states. It is expected that the wave of legalization will continue to the point of some form of federal legalization or decriminalization.

Typically, marijuana products are available to users for purchase in specialized “dispensaries” that offer dried flower, edibles, tinctures, extracts, and the like. In some cases, a unique or unusual chemical profile, or chemotype, is attractive not only for flower sales but also for use in the preparation of extracts and/or isolates and for the manufacture of a variety of products that possess characteristics of the chemotype.

### SUMMARY OF THE INVENTION

The present invention relates to a new and distinct annual variety of C. sativa, which has been given the variety denomination of ‘OT-1’. ‘OT-1’ is intended for use as medical oil, and medicinal herb for sale in Cannabis dispensaries and as a home garden plant. Plants of the new cultivar were developed to remove negative effects such as fatigue, increased appetite, anxiety, memory loss, etc., that are experienced by a consumer of the plant.

‘OT-1’ originated in Burlington Wash., in 2005. The parents are of an unknown strain derived from “bagseed” found in flower purchased for consumption. It is believed to be related to ‘Mendo Purps’.

The variety was originally grown from a seed and then cloned. The clones were self-crossed for 13 generations, and offspring of those crosses were selected for efficacy in ameliorating memory loss. Several sibling crosses within a generation were conducted using the same selection criteria, seeking to combine the favorable effects of each. Each generation produced approximately 50-150 plants which were screened. At certain stages, when a combination of desired traits was achieved in a given plant, such plant was backcrossed to a parental or grandparental generation to obtain higher expression.

The variety was propagated asexually and with seeds between 2012 and 2018 and showed a uniformity in the population. Asexual reproduction of the new variety has demonstrated that the new variety reproduces true to type with all of the morphological characteristics, as herein described, firmly fixed and retained through successive generations of such asexual propagation. Chemotypic characteristics of each new variety are variable based upon cultivation conditions, as is typical of Cannabis plants. Accordingly, while chemotypic information provided herein is representative of performance of the new varieties under a particular set of cultivation conditions, it is not limiting on other chemotypic profiles obtainable under a different set of cultivation conditions. A stable seed line of each of the new varieties has also been developed by successive generations of interbreeding and selection to correspond to the phenotypic characteristics described herein. A stable seed line of the new variety has also been developed by successive generations of interbreeding and selection to correspond to the phenotypic characteristics described herein.

TABLE 1
Exemplary Profiles of Key Cannabinoids.

| | Percent | Percent | Percent |
|---|---|---|---|
| THCA | 11.456 | 14.32 | 17.184 |
| Total THC* | 10.312 | 12.89 | 15.468 |
| Total CBD | 0.04 | 0.05 | 0.06 |
| d9-THC | 0.264 | 0.33 | 0.396 |
| CBGA | 0.728 | 0.91 | 1.092 |
| Total | 12.528 | 15.66 | 18.792 |
| Cannabinoid | | | |

*Total THC = (THCA * 0.877) + THC (i.e. delta 9 THC) + delta 8 THC

TABLE 2
Exemplary Profiles of Key Terpenes.

| | Percent | Percent | Percent |
|---|---|---|---|
| Alpha Pinene | 0.0408 | 0.051 | 0.0612 |
| Fenchol | 0.0544 | 0.068 | 0.0816 |
| Terpineol | 0.044 | 0.055 | 0.066 |
| Alpha Humulene | 0.2016 | 0.252 | 0.3024 |
| (−)-Caryophyllene | 0.0184 | 0.023 | 0.0276 |
| Ocimene | 0.0336 | 0.042 | 0.0504 |
| Eucalyptol | 0.16 | 0.20 | 0.24 |
| Phytol | 0.0688 | 0.086 | 0.1032 |
| beta Myrcene | 0.0248 | 0.031 | 0.0372 |
| Limonene | 0.38 | 0.475 | 0.57 |
| Linalool | 0.0184 | 0.023 | 0.0276 |
| beta Caryophyllene | 0.392 | 0.490 | 0.588 |
| (−)-alpha Bisabolol | 0.0536 | 0.067 | 0.0804 |
| beta Pinene | 0.06 | 0.075 | 0.09 |

### BRIEF DESCRIPTION OF THE DRAWINGS

The accompanying photographic illustrations show the typical appearance of the new variety ‘OT-1’. The colors are as nearly true as is reasonably possible in a color representation of this type. Colors in the photographs may differ slightly from the color values cited in the detailed botanical description which accurately describes the colors of the new plant.

FIG. 1 is photograph of the plant at 6 weeks of age.

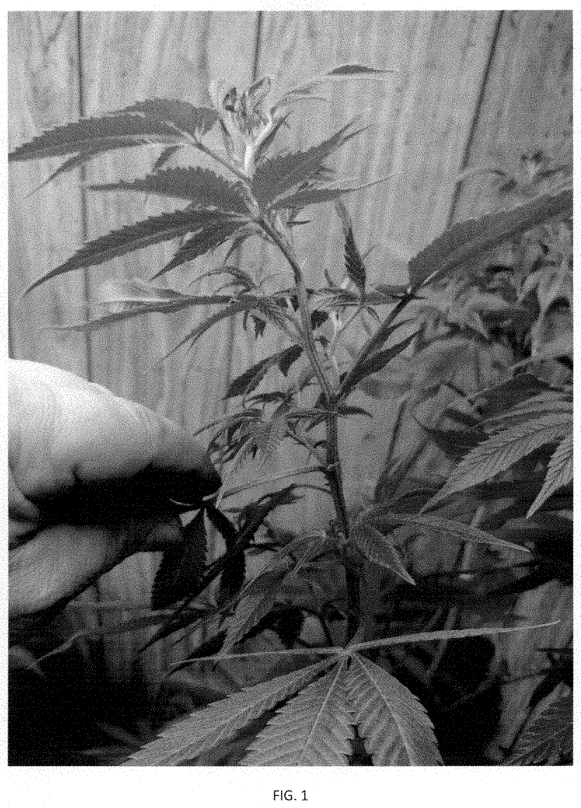

FIG. 2 is photograph of the plant at 6 weeks of age.

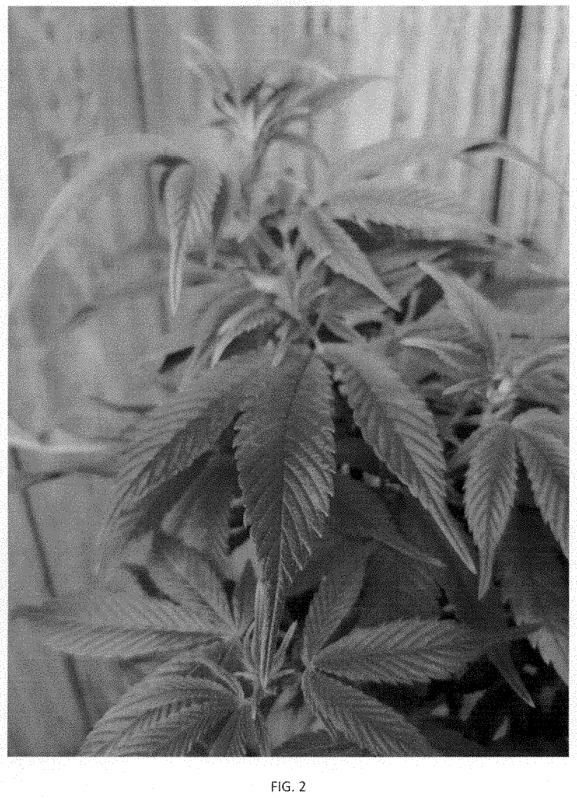

FIG. 3 is photograph of the plant at 6 weeks of age.

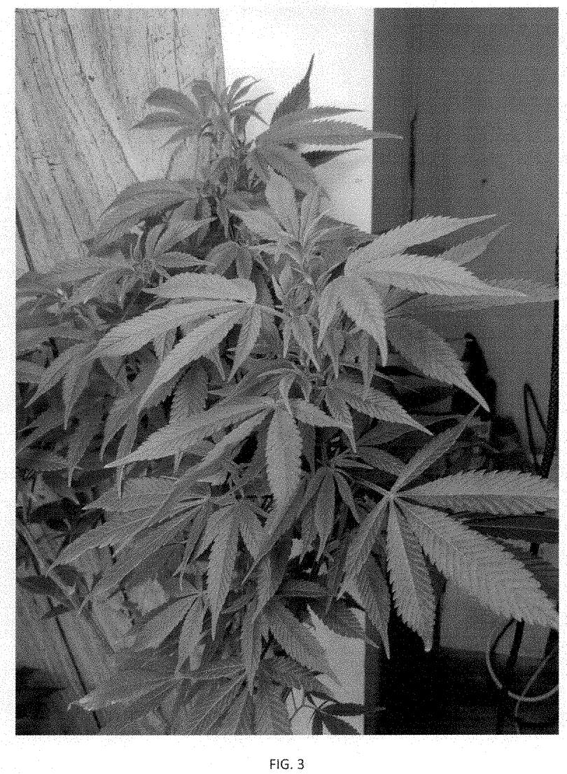

FIG. 4 is photograph of the plant at 6 weeks of age.

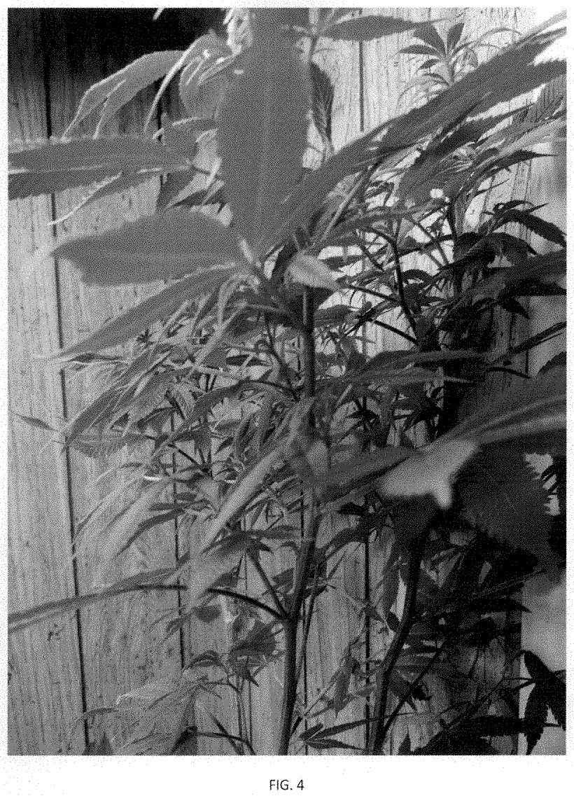

FIG. 5 is photograph of the plant at 6 weeks of age.

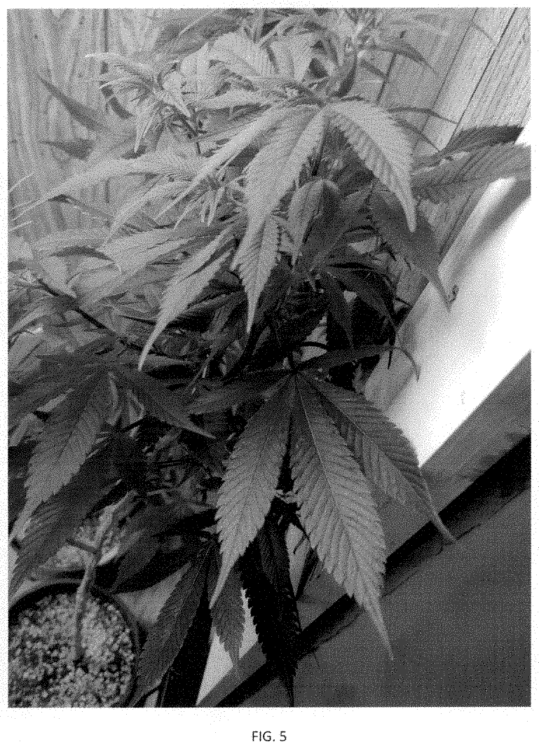

### DETAILED BOTANICAL DESCRIPTION

The following detailed description sets forth the distinctive characteristics of ‘OT-1’. The data which defines these characteristics was collected from asexual reproductions of the original selection. Dimensions, sizes, colors, and other characteristics are approximations and averages set forth as accurately as possible.
Type: Herbaceous annual.
Classification: a. Family—Cannabaceae. b. Genus—Cannabis. c. Species—sativa. d. Common Name—Marijuana.

1. A new and distinct variety of Cannabis plant named ‘OT-1’, as illustrated and described herein.
